(19) United States
(12) Patent Application Publication
(10) Pub. No.: US 2009/0221649 A1
(43) Pub. Date: Sep. 3, 2009

(54) Use of substituted 2-thio-3,5-dicyano-4-phenyl-6-aminopyridines for the treatment of reperfusion injury and reperfusion damage

(75) Inventors: Thomas Krahn, Hagen (DE); Thomas Krämer, Wuppertal (DE); Ulrich Rosentreter, Binnen (DE); James M. Downey, Mobile, AL (US); Natalia Solenkova, Nashville, TN (US)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealthCare AG, Leverkusen (DE)

(21) Appl. No.: 11/887,062

(22) PCT Filed: Mar. 11, 2006
(86) PCT No.: PCT/EP2006/002263

§ 371 Date: Dec. 15, 2008

Related U.S. Application Data
(60) Provisional application No. 60/665,631, filed on Mar. 24, 2005.

(51) Int. Cl. A61K 31/44; C07D 211/72; A61P 9/00

(52) U.S. Cl. 514/349; 546/297

(57) ABSTRACT

The invention relates to substituted 2-thio-3,5-dicyano-4-phenyl-6-aminopyridines of formula (I) and their use in medicaments for the prophylaxis and/or treatment of reperfusion injury and damage.

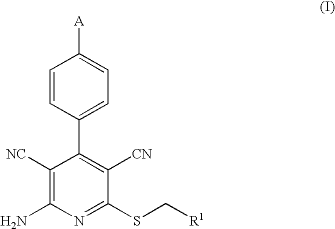

The present invention refers to the use of substituted 2-thio-3,5-dicyano-4-phenyl-6-aminopyridines of formula (I) for the production of a pharmaceutical for the prophylaxis and/or treatment of reperfusion injury and reperfusion damage.

Reperfusion injury occurs commonly after the termination of a longer lasting ischemic period, e.g. as a result of invading accumulated toxic metabolites after the reconstitution of the blood flow and/or the massive discharge of calcium ions in excitable cells. These damages occur frequently after vascular obliteration, especially acute arterial obliteration, if a compensating collateral circulation is missing (so-called infarcts). The best known forms are heart infarcts and brain infarcts (stroke). While early restoration of blood flow by thrombolysis or following transient ischemia can prevent or mitigate the degree of cell death (infarct size), reperfusion can still result in some degree of cardiac dysfunction or cell death. Thus, it would be of great clinical value to find a means to preserve normal function of the heart during reperfusion and during various forms of cardiac surgery.

Ischemia-reperfusion injury and cellular damage is known to occur in, but not limited to, myocardial infarction, coronary artery bypass grafting, angioplastic surgery, especially open heart surgery, angina, peripheral vascular disease, stroke, tissue and organ transplants (e.g. heart, liver, kidney, lung), general surgery, acute renal failure and organ hypofusion (e.g. lung, heart, liver, intestine, pancreas, kidney, limb or brain).

It is well known, that adenosine itself and adenosine analogs like NECA (5′-N-ethylcarboxamido adenosine) in general lead to a reduction of reperfusion injury, if the treatment with these compounds starts before or sometimes during the ischemic period. Application before an ischemic period is commonly known as protection and/or preconditioning and includes cell protection, especially the protection of excitable cells (e.g. nerve and muscle cells).

Adenosine mediates its physiological effects via activation of four different receptor subtypes, A1, A2a, A2b and A3. The activation of A1 and/or A3 receptor subtypes leads to the well described protection against reperfusion damage, if the A1 and/or A3 receptor subtypes are activated before the ischemic period. Activation of A2 receptor subtypes leads, because of its vessel dilating effects, to an increase in blood flow. With adenosine itself, a reduction in infarct size has been shown in clinical studies AMISTAD I and II. The mixed A1/A2 agonist AMP 579 also showed a limitation of infarct size in rabbit hearts, if the treatment started shortly before the termination of the ischemic period (Xu Z. et al., J Mol Cell Cardiol 32, 2000). Most other experiments could not show a reduction of infarct size or reperfusion damage if administered with or after the onset of reperfusion. Postconditioning as a cardioprotective intervention has recently been reported (Zhao Z. Q. et al., Am J Physiol 285, 2003). The underlying molecular pathway of these treatment options is described by Downey and Cohen (Circulation 111, 2005).

Surprisingly, it has now been found that specific as well as non-specific non-adenosine analog adenosine agonists are suitable for the production of pharmaceuticals for the prophylaxis and/or treatment of reperfusion injury and limitation of reperfusion damage in mammals, especially in humans.

This applies particularly to compounds of formula (I), whose preparation and use as pharmaceuticals, especially for the treatment of vascular diseases, is described in WO 01/25210 and WO 03/008384.

Compounds of the formula (I) display A2b-specific effects (adenosine A2b-agonistic effect greater than a factor of 10 in comparison to the agonistic effects on the other adenosine receptor subtypes A1, A2a and A3) as well as A2b non-specific effects (at least one additional agonistic effect on one of the other adenosine receptor subtypes A1, A2a or A3, which is less than a factor of 10 different to the A2b-agonistic effect).

Subject of the present invention is therefore the use of compounds of formula (I)

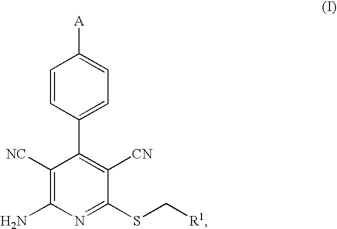

in which
A represents —O—R² or —NH—C(═O)—R³,
R¹ represents CH₁₂—C(═O)—NH₂, pyridyl or thiazolyl,
R² represents hydrogen or (C₃-C₆)-cycloalkylmethyl,
   and
R³ represents (C₁-C₄)-alkyl, (C₁-C₄)-alkoxy, mono- or di-(C₁-C₄)-alkylamino,
   and their salts, hydrates, hydrates of the salts and solvates
   for the production of a pharmaceutical for the prophylaxis and/or treatment of reperfusion injury and reperfusion damage.

Preferred according to the invention is the use of compounds of formula (I),

in which
A represents —O—R² or —NH—C(═O)—R³,
R¹ represents CH₂—C(═O)—NH₂, pyridyl or thiazolyl,
R² represents hydrogen or cyclopropylmethyl,
   and
R³ represents methyl, ethyl, n-propyl, isopropyl, n-butyl, sec-butyl, isobutyl or tert.-butyl,
   and their salts, hydrates, hydrates of the salts and solvates.

Particular preference is given to the compound of the following formula (I-A) which corresponds to Example A1 in WO 01/25210

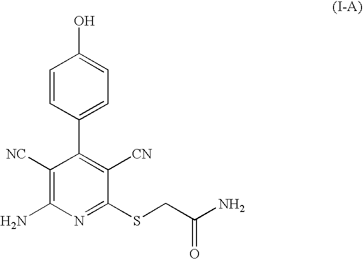

and its salts, hydrates, hydrates of the salts and solvates.

Particular preference is likewise given to the compound of the following formula (I-B)

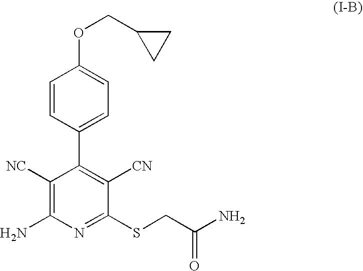

and its salts, hydrates, hydrates of the salts and solvates.

Physiologically acceptable salts are preferred in the context of this invention.

Physiologically acceptable salts according to the invention are non-toxic salts which in general are accessible by reaction of the compounds (I) with an inorganic or organic base or acid conventionally used for this purpose. Non-limiting examples of pharmaceutically acceptable salts of compounds (I) include the alkali metal salts, e.g. lithium, potassium and sodium salts, the alkaline earth metal salts such as magnesium and calcium salts, the quaternary ammonium salts such as, for example, triethyl ammonium salts, acetates, benzene sulphonates, benzoates, dicarbonates, disulphates, ditartrates, borates, bromides, carbonates, chlorides, citrates, dihydrochlorides, fumarates, gluconates, glutamates, hexyl resorcinates, hydrobromides, hydrochlorides, hydroxynaphthoates, iodides, isothionates, lactates, laurates, malates, maleates, mandelates, mesylates, methylbromides, methylnitrates, methylsulphates, nitrates, oleates, oxalates, palmitates, pantothenates, phosphates, diphosphates, polygalacturonates, salicylates, stearates, sulphates, succinates, tartrates, tosylates, valerates, and other salts used for medicinal purposes.

Hydrates of the compounds of the invention or their salts are stoichiometric compositions of the compounds with water, such as for example hemi-, mono-, or dihydrates.

Solvates of the compounds of the invention or their salts are stoichiometric compositions of the compounds with solvents.

The present invention includes both the individual enantiomers or diastereomers and the corresponding racemates or diastereomeric mixtures of the compounds according to the invention and their respective salts. In addition, all possible tautomeric forms of the compounds described above are included according to the present invention. The diastereomeric mixtures can be separated into the individual isomers by chromatographic processes. The racemates can be resolved into the respective enantiomers either by chromatographic processes on chiral phases or by resolution.

In the context of the present invention, the substituents, if not stated otherwise, in general have the following meaning:

Alkyl in general represents a straight-chain or branched hydrocarbon radical having 1 to 4 carbon atoms. Non-limiting examples include methyl, ethyl, n-propyl, isopropyl, n-butyl, isobutyl, sec.-butyl and tert.-butyl. The same applies to radicals such as alkoxy and alkylamino.

Alkoxy illustratively and preferably represents methoxy, ethoxy, n-propoxy, isopropoxy and tert.-butoxy.

Cycloalkyl in general represents a cyclic saturated hydrocarbon radical having 3 to 6 carbon atoms. Non-limiting examples include cyclopropyl, cyclobutyl, cyclopentyl and cyclohexyl.

Alkylamino represents an alkylamino radical having one or two (independently selected) alkyl substituents, illustratively and preferably representing methylamino, ethylamino, n-propylamino, isopropylamino, tert.-butylamino, N,N-dimethylamino, N,N-diethylamino, N-ethyl-N-methylamino, N-methyl-N-n-propylamino, N-isopropyl-N-n-propyl amino and N-tert.-butyl-N-methylamino.

An additional embodiment of the present invention are compounds of formula (I-C)

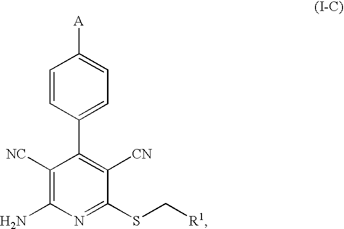

in which
A represents —O—R²,
R¹ represents CH₂—C(═O)—NH₂, pyridyl or thiazolyl,
   and
R² represents (C₃-C₆)cycloalkylmethyl,
   and their salts, hydrates, hydrates of the salts and solvates.

Preferred according to the invention are compounds of formula (I-C),

in which
A represents —O—R²,
R¹ represents CH₂—C(═O)—NH₂, pyridyl or thiazolyl,
   and
R² represents cyclopropylmethyl,
   and their salts, hydrates, hydrates of the salts and solvates.

Particular preference is given to the compound of the following formula (I-B)

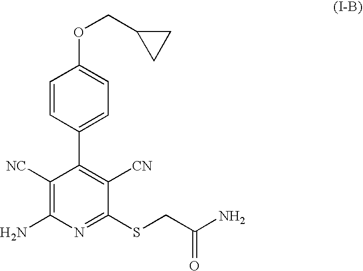

and its salts, hydrates, hydrates of the salts and solvates.

An additional embodiment of the present invention relates to a procedure for prophylaxis and/or treatment of reperfusion injury and reperfusion damage using a compound of formula (I).

An additional embodiment of the present invention is a pharmaceutical composition, comprising at least one compound according to formula I-C and/or I-B and customary auxiliaries and additives.

An additional embodiment of the present invention is a method for preparing a medicament comprising at least one compound according to formula I-C and/or I-B, wherein the active compounds are converted into a suitable administration form using customary auxiliaries and additives.

In the clinical setting and the pharmacological treatment, the administration after the onset of ischemia is the preferred practice, especially in combination with a reperfusion therapy, which has the goal to eliminate the vascular obliteration. This is independent from the fact if the vascular obliteration is eliminated by a surgical/mechanical and/or pharmacological procedure.

An additional embodiment of this invention is the pharmaceutical composition, containing a compound of formula (I) with a pharmaceutically acceptable carrier, for any of the therapeutic effects discussed above. The compositions may be administered alone or in combination with at least one other agent, such as a stabilizing compound. The compositions may be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin. Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For trans-mucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

In general, it has been found to be advantageous to administer the active compound(s) of the formula (I) in total amounts of about 0.01 to about 5000 mg per 24 h, preferably of about 0.5 to about 1000 mh per 24 h. If appropriate in a single dose or in the form of a plurality of individual administrations, to obtain the desired result.

However, it may be advantageous, if appropriate, to deviate from the amounts mentioned, depending on the nature and the body weight of the patient treated, on the individual response to the medicament, on the nature and severity of the disorder, on the nature of the preparation and the application, and on the time or interval at which administration takes place.

In the compositions described above, the active compounds of the formula (I) should be present in a concentration of from 0.1 to 99% by weight, preferably from 25-95% by weight in tablets and capsules and 1-50% by weight in fluid formulations of the total mixtures.

An additional embodiment of the present invention is the use of a combination of one or more compounds of formula (I) with one or more other agents. Suitable combination agents are for example other agents being used for the prophylaxis and/or treatment of infarcts and reperfusion damage. Exemplified and preferentially, thrombolytics are mentioned in this context.

### Experimental Part:

1. Limitation of Infarct Size, Reperfusion Injury and Other Reperfusion Damages in Isolated Rabbit Hearts by Administration of Adenosine A2b Agonists at Reperfusion:

The quantification of infarct size and experimental design followed the protocol described by Zhang et al., J Cardiovasc Pharmacol 42, 2003.

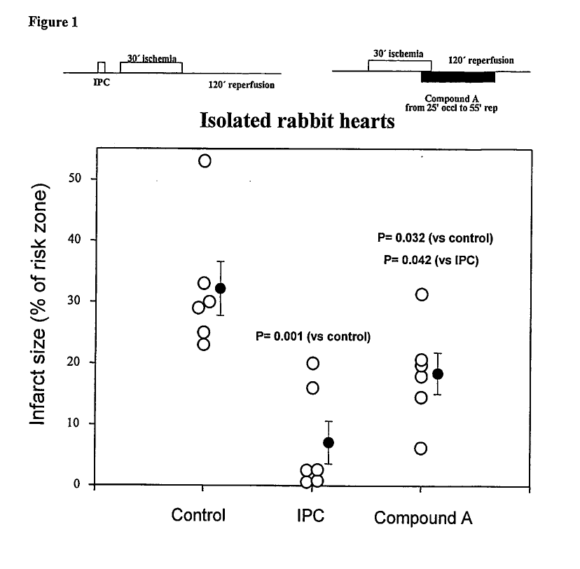

Hearts were quickly removed from anesthetized rabbits and perfused with Krebs buffer. FIG. 1 shows that the A2b-selective receptor agonist of formula (I-A) (Compound A) caused approximately a 50% reduction of infarct size in an isolated buffer-perfused rabbit heart exposed to 30 min ischemia followed by 2 hr reperfusion. The risk zone was stained with fluorescent microspheres, then the heart was sliced into 2 mm sections and the infarct size was visualized by tetrazolium staining. The drug was mixed with the perfusate at 50 μg/L starting 5 min prior to reperfusion and continuing for 55 min. The agonist was not as protective as ischemic preconditioning (IPC) with 5 min ischemia and 10 min reperfusion. Ischemic preconditioning is the most powerful cardioprotective intervention known but of no practical value clinically.

FIG. 1: Shows the infarct size in % of risk area in isolated rabbit hearts. It is also shown that the infarct size in the untreated rabbit hearts was significantly larger in comparison to the hearts treated with 50 μg/L of Compound A (p=0.032).

2. Limitation of Infarct Size, Reperfusion Injury and Other Reperfusion Damages in Rabbit Hearts (In Vivo) by Administration of Adenosine A2b Agonists at Reperfusion:

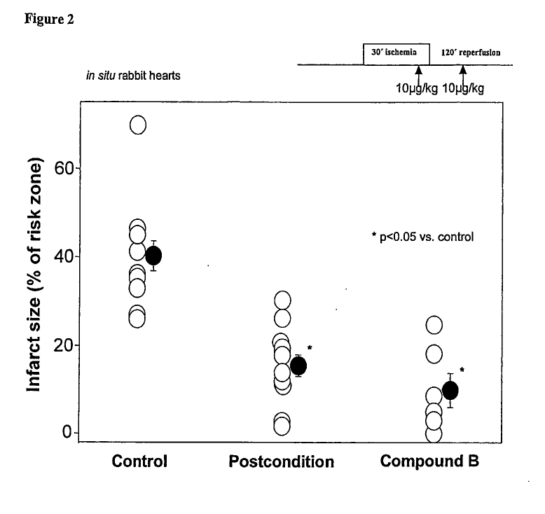

To further test the concept that an A2b receptor agonist can protect the ischemic heart, the A2b agonist of formula (I-B) (Compound B) has been examined that is well tolerated when given intravenously. FIG. 2 shows the results of Compound B given to open-chest rabbits experiencing 30 min regional ischemia and 3 hr reperfusion. Rabbits (New Zealand White rabbits of either sex weighing 1.6 to 3.0 kg) were anesthetized with sodium pentobarbital (30 mg/kg) which was subsequently supplemented as needed. Positive pressure ventilation with 100% oxygen was instituted. The heart was exposed through a left thoracotomy and a ligature was passed under a coronary branch to create the ischemia. Drug was given intravenously in a dose of 10 μg/kg over 1 min starting 5 min prior to reperfusion and again 15 min after reperfusion. The heart was removed after 3 hr of reperfusion. The risk zone was stained with fluorescent microspheres, then the heart was sliced into 2 mm sections and the infarct size was determined by tetrazolium staining.

No adverse hemodynamic effects were seen with the agent. A better than 50% reduction of infarct size was seen and was comparable to that in a third group receiving postconditioning. Postconditioning is an established cardioprotective intervention where the occluded artery is intermittently opened and closed for four 30-second cycles at the end of the ischemic insult. The A2b agonist Compound B is equivalent to postconditioning in its potency.

FIG. 2: Shows the infarct size in % of risk area in rabbit hearts in vivo. It is also shown that the treatment of rabbits with 10 μg/kg i.v. of Compound B 5 min prior and 15 min after reperfusion is as effective as postconditioning.

In conclusion an A2b receptor agonist can be effectively given to a subject at the time of reperfusion to limit myocardial infarct size.

### EXAMPLES

### Abbreviations

DCI direct chemical ionisation (for MS)

### DME N,N-dimethylformamide

DMSO dimethylsulfoxide
Hr hour(s)
Min minute(s)
MS mass spectroscopy
NMR nuclear magnetic resonance spectroscopy
of th. of theoretical (yield)

### Example 1

### 2-{[6-Amino-3,5-dicyano-4-(4-hydroxyphenyl)pyridin-2-yl]thio}acetamide (Compound A)

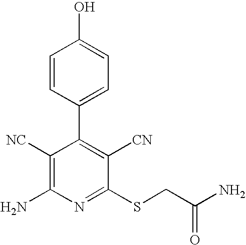

The preparation of Example 1 is described in WO 01/25210 (see Example A1).

### Example 2

### 2-({6-Amino-3,5-dicyano-4-[4-(cyclopropylmethoxy)phenyl]pyridin-2-yl}thio)acetamide (Compound B)

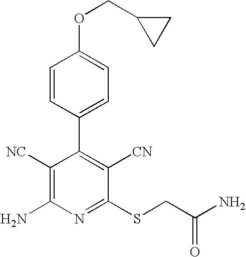

### Step 1:

### 4-(Cyclopropylmethoxy)benzaldehyde

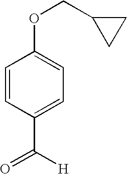

12.2 g (99.9 mmol) 4-hydroxybenzaldehyde, 13.5 g (100 mmol) (bromomethyl)cyclopropane and 13.8 g (99.8 mmol) potassium carbonate are refluxed in 200 ml acetone for 24 hr. After adding further 3.9 g (28.9 mmol) (bromomethyl)cyclopropane, the reaction mixture is refluxed for another 24 hr. After filtration and evaporation in vacuo, the residue is taken up in 50 ml ethanol and evaporated again in vacuo.

Yield: 17.6 g (100% of th.)

### Step 2:

### 4-Methylmorpholin-4-ium 6-amino-3,5-dicyano-4-[4-(cyclopropylmethoxy)phenyl]pyridine-2-thiolate

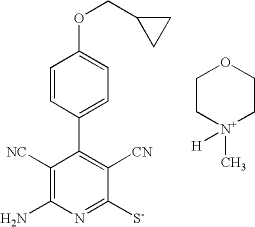

17.6 g (100 mmol) 4-(cyclopropylmethoxy)benzaldehyde, 20.3 g (200 mmol) 2-cyanoethanethioamide and 20.3 g (200 mmol) 4-methylmorpholine are refluxed in 100 ml ethanol for 3 hr. After evaporation in vacuo, the residue is taken up in 20 ml ethyl acetate and kept in a refrigerator over night. The crystals are isolated by filtration, re-suspended in little cold ethyl acetate and isolated again by filtration.

Yield: 11.2 g (26.4% of th.)

MS (DCI/NH₃): m/z=323 (M+H)

¹H-NMR (300 MHz, DMSO-d₆): δ=7.55 (broad s, 2H), 7.4 (d, 2H), 7.05 (d, 2H), 3.9 (d, 2H), 3.7 (m, 2H), 3.35 (broad s, 4H), 2.75 (s, 2H), 2.50 (s, 3H), 1.3 (m, 1H), 0.6 (m, 2H), 0.35 (m, 2H).

### Step 3:

### 2-({6-Amino-3,5-dicyano(4-[4-(cyclopropylmethoxy)phenyl]pyridin-2-yl}thio)acetamde

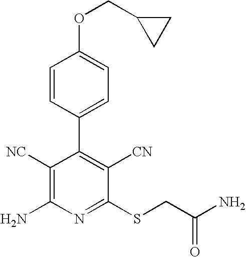

5.7 g (13.5 mmol) 4-methylmorpholin-4-ium 6-amino-3,5-dicyano-4-[4-(cyclopropylmethoxy)phenyl]pyridine-2-thiolate, 2.79 g (20.2 mmol) 2-bromoacetamide and 4.53 g (54 mmol) sodium hydrogen carbonate are stirred together in 45 ml DMF for 2 hr at room temperature. Then 22 ml methanol are added. After dropwise addition of 55 ml water, the crystals are isolated by filtration, re-suspended in water and isolated again by filtration.

Yield: 5.3 g (100% of th.)

¹H-NMR (200 MHz, DMSO-d₆): δ=8 (broad s, 2H), 7.5 (broad s, 1H), 7.45 (d, 2H), 7.25 (broad s, 1H), 7.1 (d, 2H), 3.9 (m, 4H), 1.3 (m, 1H), 0.6 (m, 2H), 0.35 (m, 2H).

1. A method for prophylaxis and/or treatment of reperfusion injury and reperfusion damage comprising administering to a subject in need thereof an effective amount of a compound of formula (I) 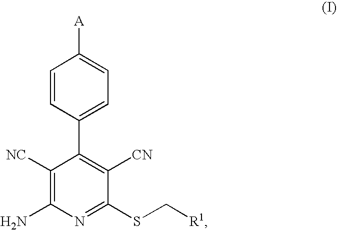
in which
A represents —O—R² or —NH—C(═O)—R³,
R¹ represents CH₂—C(═O)—NH₂, pyridyl or thiazolyl,
R² represents hydrogen or (C₃-C₆)-cycloalkylmethyl,
and
R³ represents (C₁-C₄)-alkyl, (C₁-C₄)-alkoxy, mono- or di-(C₁-C₄)-alkylamino,
and their salts, hydrates, hydrates of the salts and solvates.

2. A method according to claim 1 comprising administering to a subject in need thereof an effective amount of a compound of formula (I)
in which
A represents —O—R² or —NH—C(═O)—R³,
R¹ represents CH₂—C(═O)—NH₂, pyridyl or thiazolyl,
R² represents hydrogen or cyclopropylmethyl,
and
R³ represents methyl, ethyl, n-propyl, isopropyl, n-butyl, sec-butyl, iso-butyl or tert.-butyl,
and their salts, hydrates, hydrates of the salts and solvates.

3. A method according to claim 1 comprising administering to a subject in need thereof an effective amount of a compound of formula (I-A) 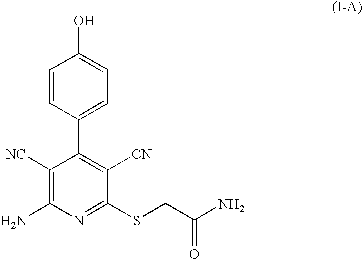
and its salts, hydrates, hydrates of the salts and solvates.

4. A method according to claim 1 comprising administering to a subject in need thereof an effective amount of a compound of formula (I-B) 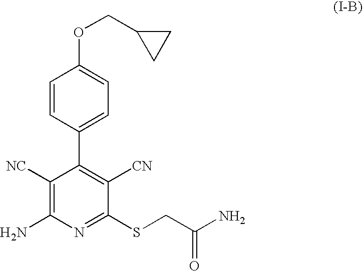
and its salts, hydrates, hydrates of the salts and solvates.

5. Compounds of formula (I-C) 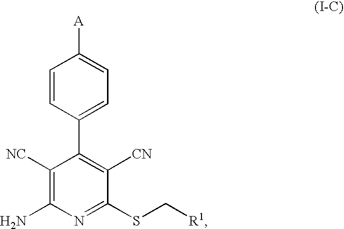
in which
A represents —O—R²,
R¹ represents CH₂—C(═O)—NH₂, pyridyl or thiazolyl,
and
R² represents (C₃-C₆)-cycloalkylmethyl,
and their salts, hydrates, hydrates of the salts and solvates.

6. Compounds according to claim 5 of formula (I-C),
in which
A represents —O—R²,
R¹ represents CH₂—C(═O)—NH₂, pyridyl or thiazolyl,
and
R² represents cyclopropylmethyl,
and their salts, hydrates, hydrates of the salts and solvates.

7. Compound according to claims 5 and 6 of the formula (I-B) 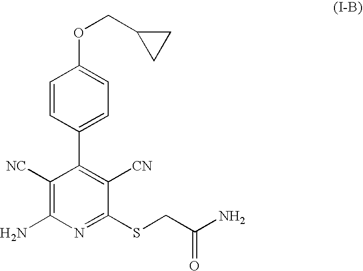
and its salts, hydrates, hydrates of the salts and solvates.

8. A pharmaceutical composition, comprising at least one compound as claimed in claims 5 to 7 and customary auxiliaries and additives.

9. A method for preparing a medicament comprising at least one compound as claimed in claim 5 to 7, wherein the active compounds are converted into a suitable administration form using customary auxiliaries and additives.

10. A method according to one of the claims 1 to 4, wherein the pharmaceutical is for oral use.

11. A method according to one of the claims 1 to 5, wherein the pharmaceutical is for prophylactic use.

12. A method for prophylaxis and/or treatment of reperfusion injury and/or reperfusion damage of a compound of the general formula (I), as defined in one of the claims 1 to 4.

13. Pharmaceutical composition for the treatment of reperfusion injury and reperfusion damage, containing a compound of the general formula (I), as defined in one of the claims 1 to 4.
